(19) United States
(12) Patent Application Publication
(10) Pub. No.: US 2017/0296616 A1
(43) Pub. Date: Oct. 19, 2017

(54) METHODS OF TREATING DEPRESSION USING NMDA MODULATORS

(71) Applicant: Naurex, Inc., Parsippany, NJ (US)

(72) Inventor: Ron Burch, Morris, CT (US)

(21) Appl. No.: 15/503,840

(22) PCT Filed: Aug. 13, 2015
(86) PCT No.: PCT/US15/45071

§ 371 Date: Feb. 14, 2017

Related U.S. Application Data
(60) Provisional application No. 62/037,374, filed on Aug. 14, 2014.

(51) Int. Cl. A61K 38/07; A61K 9/00

(52) U.S. Cl. CPC A61K 38/07; A61K 9/0019

(57) ABSTRACT

This disclosure provides methods and regimens for treating depression (e.g., treatment-resistant depression) in a patient (e.g., a patient n need of such treatment).

### CROSS REFERENCE TO RELATED APPLICATIONS

This application claims priority to U.S. provisional patent application 62/037,374 filed Aug. 14, 2014, hereby incorporated by reference in its entirety.

### BACKGROUND

Depression is a mood disorder that causes a persistent feeling of sadness and loss of interest. It is estimated that 50% or more of patients with depression do not experience an adequate therapeutic response to known administered drugs. In most instances, 2 or more weeks of drug therapy are need before meaningful improvement is observed, as noted in an open-label study on pharmacological treatment of depression. (Rush et al, Am. J. Psychiatry 2006, 163: 1905). There currently is no single effective treatment for depression, anxiety, and other related diseases.

An N-methyl-d-aspartate (NMDA) receptor (NMDAR) is a postsynaptic, ionotropic receptor that is responsive to, inter alia, the excitatory amino acids glutamate and glycine and the synthetic compound NMDA. The NMDA receptor controls the flow of both divalent and monovalent ions into the postsynaptic neural cell through a receptor associated channel (Foster et al., Nature 1987, 329:395-396; Mayer et al., Trends in Pharmacol. Sci. 1990, 11:254-260).

The NMDA receptor has been implicated during development in specifying neuronal architecture and synaptic connectivity, and may be involved in experience-dependent synaptic modifications. In addition, NMDA receptors are also thought to be involved in long term potentiation and central nervous system disorders.

The NMDA receptor is believed to consist of several protein chains embedded in the postsynaptic membrane. The first two types of subunits discovered so far form a large extracellular region, which probably contains most of the allosteric binding sites, several transmembrane regions looped and folded so as to form a pore or channel, which is permeable to Ca⁺⁺, and a carboxyl terminal region. The opening and closing of the channel is regulated by the binding of various ligands to domains (allosteric sites) of the protein residing on the extracellular surface. The binding of the ligands is thought to affect a conformational change in the overall structure of the protein which is ultimately reflected in the channel opening, partially opening, partially closing, or closing.

The NMDA receptor plays a major role in the synaptic plasticity that underlies many higher cognitive functions, such as memory acquisition, retention and learning, as well as in certain cognitive pathways and in the perception of pain (Collingridge et al., The NMDA Receptor, Oxford University Press, 1994). In addition, certain properties of NMDA receptors suggest that they may be involved in the information-processing in the brain that underlies consciousness itself.

The NMDA receptor has drawn particular interest since it appears to be involved in a broad spectrum of CNS disorders. For instance, during brain ischemia caused by stroke or traumatic injury, excessive amounts of the excitatory amino acid glutamate are released from damaged or oxygen deprived neurons. This excess glutamate binds to the NMDA receptors which opens their ligand-gated ion channels; in turn the calcium influx produces a high level of intracellular calcium which activates a biochemical cascade resulting in protein degradation and cell death. This phenomenon, known as excitotoxicity, is also thought to be responsible for the neurological damage associated with other disorders ranging from hypoglycemia and cardiac arrest to epilepsy. In addition, there are preliminary reports indicating similar involvement in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. Neuropsychiatric involvement of the NMDA receptor has also been recognized since blockage of the NMDA receptor Ca⁺⁺ channel by the animal anesthetic PCP (phencyclidine) produces a psychotic state in humans similar to schizophrenia (reviewed in Johnson, K. and Jones, S., 1990). Further, NMDA receptors have also been implicated in certain types of spatial learning.

Recent human clinical studies have identified NMDAR as a novel target of high interest for treatment of depression. These studies conducted using known NMDAR antagonists CPC-101,606 and ketamine have shown significant reductions in the Hamilton Depression Rating Score in patients suffering with refractory depression. While the efficacy was significant, the side effects of using these NDMAR antagonists were reportedly severe.

Recently, an improved partial agonist of NMDAR, termed as GLYX-13, has been reported. GLYX-13 is exemplified by the following structure:

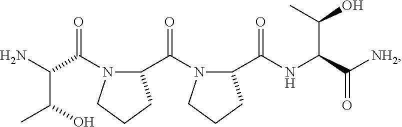

with a molecular weight: 413.47, and a chemical formula: C₁₈H₃₁N₅O₆. GLYX-13 exhibits nootropic, neuroprotective and antinociceptive activity, and enhances learning, memory and cognition in vivo. GLYX-13, has also been shown to exhibit rapid-acting, robust, and sustained antidepressant activity and to lack the pyschotomimetic side effects associated with other drugs and mechanisms that target the NMDA receptor.

### SUMMARY

This disclosure provides methods and regimens for treating depression (e.g., treatment-resistant depression) in a patient (e.g., a patient in need of such treatment), for example, a patient being treated for depression by another anti-depressant without achieving significantly full response or effectiveness to treatment on the other anti-depressant alone. Candidate patient(s) can include, without limitation, individual(s) that (i) have self-reported one or more symptoms of depression; and/or (ii) have been diagnosed as suffering from depression (e.g., untreated depression, e.g., untreated for 4, 5, 6, 7, 8 or more weeks), e.g., received a score greater than 7 on the Hamilton Depression Rating Scale (“HDRS”) and/or a score greater than 10 on the Montgomery-Asberg Depression Rating Scale (MADRS); and/or (iii) have undergone, or are currently undergoing, treatment for depression with at least one other anti-depressant; and/or (iv) are predisposed to, or at risk of, depression. The methods and regimens disclosed herein include administering a particular dose (or a range of doses) of GLYX-13 (or a composition containing GLYX-13) at a particular frequency (or a range of frequencies, e.g., weekly or once every two weeks) over a time period that is sufficient so as to provide the patient with two or more (e.g., two, three, four, five, six, seven, eight, nine, ten, eleven, twelve) doses of GLYX-13 over said time period. The period of time during which the patient receives the two or more doses is sometimes referred to herein as an “induction period of time” (also sometimes referred to herein as “repeat” or “repeated” dosing). The methods and regimens described herein can further include a “rest period of time,” during which time the patient does not receive GLYX-13 (or a composition containing the same). In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc. The methods and regimens described herein can further include administering one or more other anti-depressants at any point before, during, or after any induction period of time and/or any rest period of time.

Advantageously, the methods and regimens disclosed herein result in long lasting efficacy without producing unwanted side effects. In some embodiments, the efficacy achieved at the end of an induction period is maintained during the ensuing rest period of time. In some embodiments, the efficacy achieved is reestablished (e.g., due to relapse, e.g., a forced relapse) in a relatively rapid and substantially complete manner upon administration of GLYX-13.

Accordingly, in one aspect, this disclosure features methods of stabilizing a patient being treating for depression, which include intravenously administering to the patient an effective amount of a composition comprising GLYX-13, wherein the composition is administered to the patient once every week or once every two weeks for an induction period of time. In some embodiments, a patient being treated for depression is being administered another anti-depressant without achieving full response on the other anti-depressant alone.

In another aspect, this disclosure features methods of treating depression in a patient need thereof, comprising sequentially administering to the patient between about 5 mg/kg and about 10 mg/kg of GLYX-13, or for a first period of time, and wherein the sequentially administrating is followed by not administering GLYX-13 for a rest period of time of time.

In a further aspect, this disclosure features regimens for treating depression in a human patient, which include delivering to the patient GLYX-13 in a cycle of treatment, said cycle comprising intravenously administering a dosage of about 5 mg/kg to about 10 mg/kg of GLYX-13 (or about 2.5 mg/kg to about 10 mg/kg of GLYX-13, or for example, about 225 mg to about 900 mg of GLYX-13) per week or every other week for at least four weeks in the cycle followed by at least one week, two weeks, three weeks, four weeks, two months or more where no GLYX-13 is administered.

### BRIEF DESCRIPTION OF THE DRAWINGS

FIG. 1 is a graph showing improvements (based on HDRS scale) achieved during an induction period of time (identified as “stabilization” on the graph) are generally maintained throughout the rest period of time (identified as “randomized withdrawal” on the graph), although HDRS scores did vary with dosage frequency.

FIG. 2 is a graph showing a sub-set of data from the graph shown in FIG. 1 and shows that data for subject with drug withdrawn appear to show long-lasting effect of GLYX-13.

FIG. 3 is a series of graphs showing that GLYX-13 metaplasticity enhances long-term potentiation (“LTP”) 24 hours and one week following a single dose, and persistently enhances LTP following multiple bi-weekly doses.

FIG. 4 is a graph showing improvements (based on Bech-6 scale) achieved during an induction period of time (identified as “stabilization” on the graph) are generally maintained throughout the rest period of time (identified as “randomized withdrawal” on the graph), although Bech-6 scores did vary with dosage frequency.

FIG. 5 is a graph showing improvements (based on CGI-S scale) achieved during an induction period of time are generally maintained throughout the rest period of time (identified as “Week 7” and “Week 13” on the graph), although CGI-S scores did vary with dosage frequency.

FIG. 6 is a graph indicated an example of stabilization phase of GLYX-13 if reestablished following forced relapse. Repeated crossover can demonstrate repeated response to GLYX-13 and relapse to placebo.

FIGS. 7A and 7B show typical design of randomized withdrawal maintenance study.

### DETAILED DESCRIPTION

This disclosure provides methods and regimens for treating depression (e.g., treatment-resistant depression) in a patient (e.g., a patient in need of such treatment). Candidate patient(s) can include, without limitation, individual(s) that (i) have self-reported one or more symptoms of depression; and/or (ii) have been diagnosed as suffering from depression (e.g., untreated depression, e.g., untreated for 4, 5, 6, 7, 8 or more weeks), e.g., received a score greater than 7 on the Hamilton Depression Rating Scale (“HDRS”) and/or a score greater than 10 on the Montgomery-Asberg Depression Rating Scale (MADRS); and/or (iii) have undergone, or are currently undergoing, treatment for depression with at least one other anti-depressant; and/or (iv) are predisposed to, or at risk of, depression. The methods and regimens disclosed herein include administering a particular dose (or a range of doses) of GLYX-13 (or a composition containing GLYX-13) at a particular frequency (or a range of frequencies, e.g., weekly or once every two weeks) over a time period that is sufficient so as to provide the patient with two or more (e.g., two, three, four, five, six, seven, eight, nine, ten, eleven, twelve) doses of GLYX-13 over said time period. The period of time during which the patient receives the two or more doses is sometimes referred to herein as an “induction period of time” (also sometimes referred to herein as “repeat” or “repeated” dosing). The methods and regimens described herein can further include a “rest period of time,” during which time the patient does not receive GLYX-13 (or a composition containing the same). In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc. The methods and regimens described herein can further include administering one or more other anti-depressants at any point before, during, or after any induction period of time and/or any rest period of time.

### Depression

Depression is a common psychiatric disorder and refers to a mental state of low mood and aversion to activity. Various symptoms associated with depression include persistent anxious or sad feelings, feelings of helplessness, hopelessness, pessimism, and/or worthlessness, low energy, restlessness, irritability, fatigue, loss of interest in pleasurable activities or hobbies, excessive sleeping, overeating, appetite loss, insomnia, thoughts of suicide, and suicide attempts. The presence, severity, frequency, and duration of the above mentioned symptoms vary on a case to case basis. In some embodiments, a patient may have at least one, at least two, at least three, at least four, or at least five of these symptoms.

The most common depression conditions include Major Depressive Disorder and Dysthymic Disorder. Other depression conditions develop under unique circumstances. Such depression conditions include but are not limited to Psychotic depression, Postpartum depression, Seasonal affective disorder (SAD), mood disorder, depressions caused by chronic medical conditions such as cancer or chronic pain, chemotherapy, chronic stress, post-traumatic stress disorders, and Bipolar disorder (or manic depressive disorder).

Treatment resistant depression (sometimes referred to herein as refractory depression) occurs in patients suffering from depression who are resistant to standard pharmacological treatments, including tricyclic antidepressants, MAOIs, SSRIs, esketamine or other NMDA modulators, double and triple uptake inhibitors and/or anxiolytic drugs, and anti-psychotic treatments, as well non-pharmacological treatments such as psychotherapy, electroconvulsive therapy, vagus nerve stimulation and/or transcranial magnetic stimulation. A treatment resistant-patient may be identified as one who fails to experience alleviation of one or more symptoms of depression (e.g., persistent anxious or sad feelings, feelings of helplessness, hopelessness, pessimism) despite undergoing one or more standard pharmacological or non-pharmacological treatment. In certain embodiments, a treatment-resistant patient is one who fails to experience alleviation of one or more symptoms of depression despite undergoing treatment with two different antidepressant drugs. In other embodiments, a treatment-resistant patient is one who fails to experience alleviation of one or more symptoms of depression despite undergoing treatment with three or four different antidepressant drugs. A treatment-resistant patient may also be identified as one who is unwilling or unable to tolerate the side effects of one or more standard pharmacological or non-pharmacological treatment.

“Treating” includes any effect, e.g., lessening, reducing, modulating, or eliminating, that results in the improvement of the condition, disease, disorder and the like. “Individual,” “patient,” or “subject” are used interchangeably and include any animal, including mammals, preferably mice, rats, other rodents, rabbits, dogs, cats, swine, cattle, sheep, horses, or primates, and most preferably humans.

The term “effective amount” refers to an amount of the subject component, e.g., GLYX-13 (or a composition containing GLYX-13) that will elicit the biological or medical response of a tissue, system, animal or human that is being sought by the researcher, veterinarian, medical doctor or other clinician. Therapeutically effective amounts of GLYX-13 (or a composition containing GLYX-13), as well as any other antidepressant used in combination with GLYX-13, can each vary with the form of the depression condition being treated, the length of treatment time desired, the age and the condition of the patient, and is ultimately determined by the attending physician. By way of example, an effective amount can be an amount effective to treat any of the diseases, disorders, and conditions described herein (e.g., treatment-resistant depression). Alternatively, an effective amount can refer the quantity needed to achieve a desired therapeutic and/or prophylactic effect, e.g., during an ensuing rest period of time, the patient substantially maintains a level of improvement of depression symptoms as compared to a level of improvement achieved after an induction period of time as indicated by one or more of the following scales or measures: HDRS, MADRS, or % reduction in symptoms from baseline (e.g., as determined immediately after the induction period and during the rest period of time). By way of example, during the ensuing rest period of time, the patient substantially maintains a HDRS-17 score of less than or about 7; and/or maintains a MADRS score of less than or about 10; and/or maintains a greater than or equal to about 50% reduction in depression symptoms from baseline. Achieving and maintaining improvement of depression symptoms in a patient (e.g., achieving and maintaining a HDRS-17 score of less than or about 7; and/or a MADRS score of less than or about 10; and/or a greater than or equal to about 50% reduction in depression symptoms from baseline) is sometimes referred to herein as “stabilizing” a patient.

### GLYX-13

“GLYX-13” is represented by the following formula:

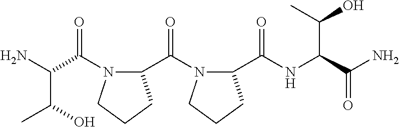

and includes polymorphs, hydrates, solvates, free bases, and/or suitable salt forms of the above compound.

GLYX-13 may be obtained by recombinant or synthetic methods such as those described in U.S. Pat. Nos. 5,763,393 and 4,086,196 herein incorporated by reference. Also contemplated are polymorphs, hydrates, homologs, solvates, free bases, and/or suitable salt forms of GLYX 13 such as, but not limited to, the acetate salt. The peptide may be in cyclized or non-cyclized form as further described in U.S. Pat. No. 5,763,393. In some embodiments, a GLYX-13 analog may include an insertion or deletion of a moiety on one or more of the Thr or Pro groups such as a deletion of CH₂, OH, or NH₂ moiety. In other embodiments, GLYX-13 may be optionally substituted with one or more halogens, C₁-C₃ alkyl (optionally substituted with halogen or amino), hydroxyl, and/or amino. Other compounds contemplated for use herein include Glycine-site partial agonists of the NMDAR disclosed in U.S. Pat. No. 5,763,393, U.S. Pat. No. 6,107,271, and Wood et al., Neuro. Report, 19, 1059-1061, 2008, the entire contents of which are herein incorporated by reference.

It may be understood that the peptides disclosed here can include both natural and unnatural amino acids, e.g., all natural amino acids (or derivatives thereof), all unnatural amino acids (or derivatives thereof), or a mixture of natural and unnatural amino acids. For example, one, two, three or more of the amino acids in GLYX-13 may each have, independently, a d- or 1-configuration.

### Methods

In one aspect, this disclosure features methods of stabilizing a patient being treating for depression, which include intravenously administering to the patient an effective amount of a composition comprising GLYX-13, wherein the composition is administered to the patient once every week or once every two weeks for an induction period of time.

In some embodiments, each induction period of time is, independently, from about one week to about six months (e.g., from about two weeks to about six months, from about three weeks to about six months, from about four weeks to about six months, from about five weeks to about six months, from about six weeks to about six months). In certain embodiments, each induction period of time is, independently, from about three weeks to about sixteen weeks, from about three weeks to about twelve weeks, from about three weeks to about ten weeks, from about three weeks to about eight weeks, from about three weeks to about six weeks; from about four weeks to about sixteen weeks, from about four weeks to about twelve weeks, from about four weeks to about ten weeks, from about four weeks to about eight weeks, from about four weeks to about six weeks; from about five weeks to about sixteen weeks, from about five weeks to about twelve weeks, from about five weeks to about ten weeks, from about five weeks to about eight weeks, from about five weeks to about six weeks; from about six weeks to about sixteen weeks, from about six weeks to about twelve weeks, from about six weeks to about ten weeks, or from about six weeks to about eight weeks.

In certain embodiments, each induction period of time is, independently, from about three weeks to about twelve weeks, e.g., from about four weeks to about twelve weeks, from about six weeks to about twelve weeks, from about five weeks to about eight weeks. In certain embodiments, each induction period of time is, independently, about three weeks, about four weeks, about five weeks, about six weeks, about seven weeks, about eight weeks, about ten weeks, or about twelve weeks, e.g., about six weeks.

In some embodiments, after the induction period of time, GLYX-13 (or a composition containing GLYX-13) is not administered to the patient for a rest period of time (or a withdrawal period of time). In some embodiments, each rest period of time is, independently, from about one week to about six months (e.g., from about one week to about sixteen weeks, from about one week to about twelve weeks, from about one week to about ten weeks, from about one week to about eight weeks, from about one week to about six weeks, from about one week to about four weeks, from about one week to about three weeks). In certain embodiments, each rest period of time is, independently, from about one week to about six weeks.

In some embodiments, each induction period of time is, independently, from about three weeks to about twelve weeks, e.g., from about four weeks to about twelve weeks, from about six weeks to about twelve weeks, from about five weeks to about eight weeks, and each rest period of time is, independently, from about one week to about six weeks. In certain embodiments, each induction period of time is, independently, about three weeks, about four weeks, about five weeks, about six weeks, about seven weeks, about eight weeks, about ten weeks, or about twelve weeks, e.g., about six weeks, and each rest period of time is, independently, from about one week to about six weeks.

In some embodiments, a therapeutically effective amount of GLYX-13 for adult human treatment administered, for example, during an induction period of time, are in the range of about 0.01 mg/kg to about 1000 mg/kg per administration (e.g., about 0.01 mg/kg to about 100 mg/kg, about 0.01 mg/kg to about 50 mg/kg, about 0.01 mg/kg to about 25 mg/kg, about 0.01 mg/kg to about 10 mg/kg, about 0.1 mg/kg to about 100 mg/kg, about 0.1 mg/kg to about 50 mg/kg, about 0.1 mg/kg to about 50 mg/kg, about 0.1 mg/kg to about 10 mg/kg, about 1 mg/kg to about 100 mg/kg, about 1 mg/kg to about 50 mg/kg, about 1 mg/kg to about 50 mg/kg per day, about 1 mg/kg to about 10 mg/kg, or about 1 mg/kg to about 10 mg/kg per administration, e.g., once a week, twice a week or three times a week and/or as described herein). The dosage of GLYX-13 may be at any dosage including, but not limited to, about 1 ug/kg, 25 ug/kg, 50 ug/kg, 75 ug/kg, 100 u ug/kg, 125 ug/kg, 150 ug/kg, 175 ug/kg, 200 ug/kg, 225 ug/kg, 250 ug/kg, 275 ug/kg, 300 ug/kg, 325 ug/kg, 350 ug/kg, 375 ug/kg, 400 ug/kg, 425 ug/kg, 450 ug/kg, 475 ug/kg, 500 ug/kg, 525 ug/kg, 550 ug/kg, 575 ug/kg, 600 ug/kg, 625 ug/kg, 650 ug/kg, 675 ug/kg, 700 ug/kg, 725 ug/kg, 750 ug/kg, 775 ug/kg, 800 ug/kg, 825 ug/kg, 850 ug/kg, 875 ug/kg, 900 ug/kg, 925 ug/kg, 950 ug/kg, 975 ug/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg. In certain embodiments, GLYX-13 may be therapeutically effective for depression with a range (e.g., an intravenous dose range) of about 1 to about 10 mg/kg, e.g., about 5 to about 10 mg/kg, e.g. about 1 mg/kg, about 5 mg/kg, or about 10 mg/kg.

In some embodiments, a therapeutically effective amount of GLYX-13 for adult human treatment administered, for example, during an induction period of time may be a fixed dose of about 1000 mg to about 200 mg, or 900 mg to about 100 mg e.g., about 200 mg to about 500 mg, e.g., 50 mg, 100 mg, 225 mg, 250 mg, 200 mg, 300 mg, 350 mg, 450 mg, 500 mg, 600 mg, 700 mg, 750 mg, and/or 900 mg unit dose. It will be appreciated that a maintenance dose may be lower than the induction dose.

In some embodiments, any of the GLYX-13 dosages described herein can be administered on a less than daily basis, e.g., every other day (e.g., every two days); one or two times a week; one, two or three times a week; two or three times a week; twice weekly (e.g. every 3 days, every 4 days, every 5 days, every 6 days or e.g. administered with an interval of about 2 to about 3 days between doses); every three to four days; once a week; once every two weeks (bi-weekly); twice monthly; once a month or even less often. In certain embodiments, GLYX-13 is administered at a frequency of once a week, twice a week, once every two weeks, or any combination thereof.

In certain embodiments GLYX-13 (rapastinel) is administered at a range (e.g., an intravenous dose range) of about 1 to about 10 mg/kg, e.g., about 5 to about 10 mg/kg, e.g. about 1 mg/kg, about 5 mg/kg, or about 10 mg/kg, and/or GLYX-13 is administered at a frequency of once a week, once every two weeks, or any combination thereof.

By way of example, methods of treating depression in a patient need thereof can include sequentially administering to the patient between about 2.5 mg/kg and about 10 mg/kg, or about 5 mg/kg and about 10 mg/kg of GLYX-13 (or about 225 mg to about 900 mg of GLYX-13) for a first period of time (i.e., an induction period of time), and wherein the sequentially administrating is followed by not administering GLYX-13 for a rest period of time of time. In certain embodiments, the sequentially administrating and not administering are repeated at least once (e.g., twice, three times, four times, five times, six times, seven times, eight times, nine times, ten times, eleven times, twelve times). In certain embodiments, the GLYX-13 is administered weekly or every other week for a first period of time (i.e., an induction period of time) of about three weeks to about twelve weeks (e.g., sequentially administering about 5 mg/kg or about 10 mg/kg weekly or every other week for three to twelve weeks, five to ten weeks, three to six weeks, six to twelve weeks or more), and wherein the rest period of time is about one to about six weeks (or more), e.g. one, two three, four, five, six, seven or eight weeks or more.

In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc.

By way of example, regimens for treating depression in a human patient can include delivering to the patient GLYX-13 in a cycle of treatment, in which the cycle intravenously administering a dosage of about 5 mg/kg to about 10 mg/kg of GLYX-13 per week or every other week for at least four weeks in the cycle (i.e., an induction period of time) followed by at least one week, two weeks, three weeks, four weeks, two months or more where no GLYX-13 is administered (rest period of time). In certain embodiments, the cycle's induction period of time includes at least three weekly dosage administrations. In certain embodiments, the cycle is repeated at least once (e.g., twice, three times, four times, five times, six times, seven times, eight times, nine times, ten times, eleven times, twelve times). In certain embodiments, the cycles are continuous.

In some embodiments, patient(s) can include, without limitation, individual(s) that (i) have self-reported one or more symptoms of depression; and/or (ii) have been diagnosed as suffering from depression (e.g., untreated depression, e.g., untreated for 4, 5, 6, 7, 8 or more weeks), e.g., received a score greater than 7 on the Hamilton Depression Rating Scale (“HDRS”) and/or a score greater than 10 on the Montgomery-Asberg Depression Rating Scale (MADRS); and/or (iii) have undergone, or are currently undergoing, treatment for depression with at least one (e.g., at least two, at least three) other anti-depressant(s); and/or (iv) are predisposed to, or at risk of, depression. In certain embodiments, the patient is a treatment-resistant patient (e.g., identified as one who has been treated with at least one type of antidepressant treatments prior to administration of GLYX-13 or one who has been treated with at least two types of antidepressant treatments prior to administration of GLYX-13).

In some embodiments, the depression is selected from the group consisting of major depressive disorder, dysthymic disorder, psychotic depression, postpartum depression, seasonal affective disorder, bipolar disorder, bipolar depression, mood disorder, depressions caused by chronic medical conditions such as cancer or chronic pain, chemotherapy, chronic stress, post-traumatic stress disorders, and manic depressive disorder. In certain embodiments, the depression is treatment-resistant depression.

### Combination Therapy

The present disclosure contemplates “combination therapy,” which includes (but is not limited to) co-administering an effective amount of GLYX-13 and one or more other biologically active agents (e.g., one or more other anti-depressant agents) as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. The methods and regimens described herein can further include administering one or more other anti-depressants at any point before, during, or after any induction period of time and/or any rest period of time. Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule or i.v. solution having a fixed ratio of each therapeutic agent or in multiple, single tablets, capsules, or i.v. solutions for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent (e.g., GLYX-13) of the combination selected may be administered by intravenous injection while the other therapeutic agents of the combination may be administered orally. Alternatively, for example, all therapeutic agents may be administered orally or all therapeutic agents may be administered by intravenous injection.

In some embodiments, GLYX-13 in administered in combination with one or more other antidepressant treatments, such as, tricyclic antidepressants, MAO-I's, SSRI's, SNRI's and double and triple uptake inhibitors, atypical antipsychotics, and/or anxiolytic drugs for manufacturing a medicament for treating depression, anxiety, and/or other related diseases including provide relief from depression, anxiety and preventing recurrence of depression and anxiety. Exemplary drugs that may be used in combination with a GLYX peptide include Anafranil, Adapin, Aventyl, Buproprion, Elavil, Norpramin, Pamelor, Pertofrane, Sinequan, Surmontil, Tofranil, Vivactil, Parnate, Nardil, Marplan, Celexa, Lexapro, Luvox, Paxil, Prozac, Zoloft, Wellbutrin, Effexor, Remeron, Cymbalta, Desyrel (trazodone), and Ludiomill. It will be appreciated that in some embodiments, administration of GLYX-13 may act more quickly than a co-administered antidepressant treatment, and thus such co-administration (e.g., administration of GLYX-13 on an acute or immediate basis, while starting a regimen with another, slower acting anti-depressant at about the same time) may be particularly advantageous in the common situation where the second antidepressant is slower acting. In some embodiments, GLYX-13 in administered in combination with one or more NMDAR antagonists. In certain embodiments, the disclosed compound, e.g. GLYX-13, may be dosed at amount that reverses or prevents cognitive impairment. In some embodiments, the NMDAR antagonist is selected from the group consisting of ketamine, esketamine, memantine, lanicemine (AZD6765), CERC-301, dextromethorphan, dextrorphan, phencyclidine, dizocilpine (MK-801), amantadine, ifenprodil, and riluzole, or a pharmaceutically acceptable salt or prodrug thereof. Also contemplated are derivatives of the aforementioned NMDAR antagonists.

Also contemplated herein are methods of treating depression that include administering GLYX-13 in combination with (e.g. simultaneously or sequentially) other non-pharmacological treatments such as psychotherapy, electroconvulsive therapy, vagus nerve stimulation and/or transcranial magnetic stimulation.

In certain embodiments patient is administered one or more other pharmacological and/or non-pharmacological treatments (e.g., one or more other antidepressant agents) before and/or during or after the induction period of time. In other embodiments, the patient is administered one or more other pharmacological and/or non-pharmacological treatments (e.g., one or more other antidepressant agents) during the rest period of time. In still other embodiments the patient is administered one or more other pharmacological and/or non-pharmacological treatments (e.g., one or more other antidepressant agents) before and/or during or after the induction period of time, and the patient is administered one or more other pharmacological and/or non-pharmacological treatments (e.g., one or more other antidepressant agents) during the rest period of time. Accordingly, it is understood that a patient can receive any of the above-described other pharmacological and/or non-pharmacological treatments before and/or during any treatment cycle.

### Administration and Formulations

GLYX-13 as well as any other pharmacological agent (e.g., one or more other antidepressant agents) of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations, or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In some embodiments, GLYX-13 herein may be administered parenterally to a patient including, but not limited to, subcutaneously, intramuscularly, and intravenously. In some embodiments, one or more of the components of the combinations described herein may also be administered via slow controlled i.v. infusion or by release from an implant device.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, intranasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards. The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The combinations described herein may also contain other active compounds providing supplemental, additional, or enhanced therapeutic functions. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

Disclosed compounds may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The present invention has multiple aspects, illustrated by the following non-limiting examples.

### EXAMPLES

### Example 1 Repeat Dose Study

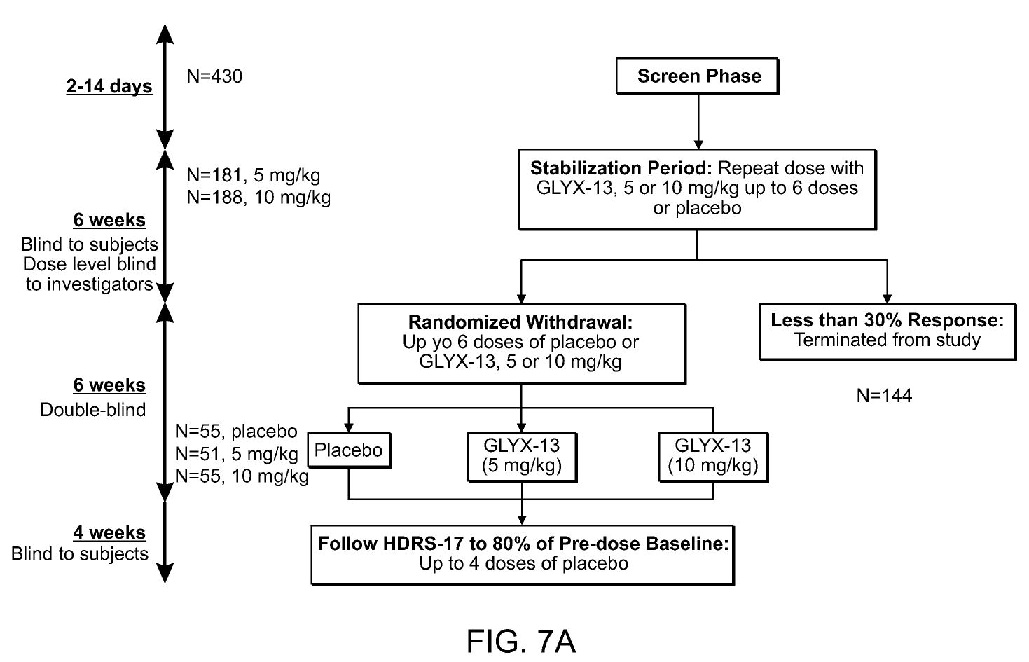

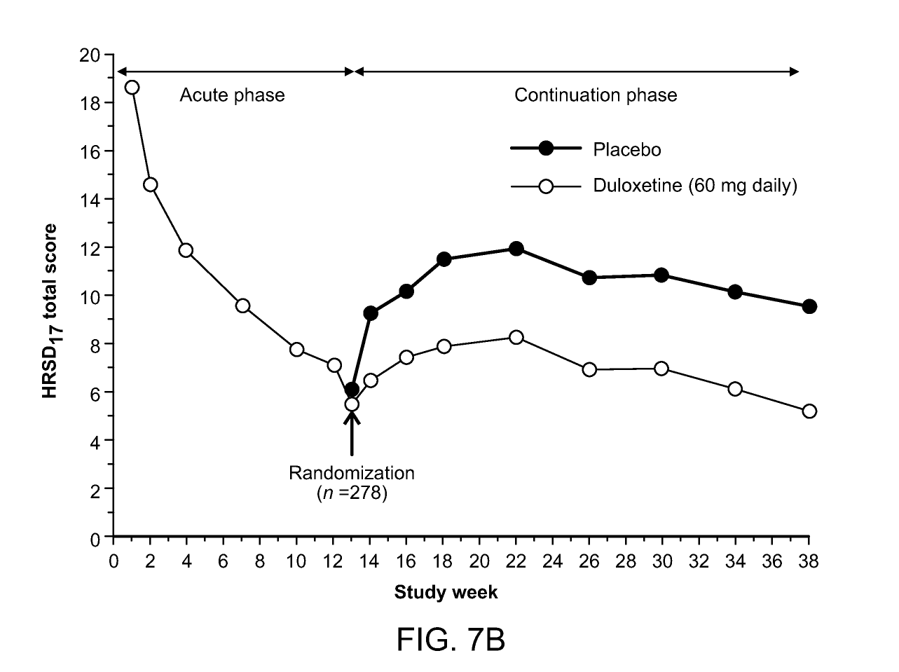

A repeat dose study was conducted on human subjects using adjunctive dosing (patients were currently taking another antidepressant medication). Dose levels of 5 mg/kg and 10 mg/kg were used with weekly or biweekly dose during an initial, stabilization period, as shown in FIGS. 7A and 7B (showing typical design of randomized withdrawal maintenance study). The disposition of the subjects, as indicated in FIGS. 7A and 7B was 144 nonresponders and 164 responders, where of the responders (70 patients assigned to weekly dosing, 36 patients assigned to biweekly dosing, 55 patients randomized to placebo). During the stabilization phase, most responders identified with few doses, e.g., ˜65% of ultimate responders showed response after one dose, ˜72% of ultimate responders showed response after two doses, and ˜85% of ultimate responders showed response after three doses. The study was blinded to third party raters (HDRS-17) and to clinicians of the treatment group (CGI-S).

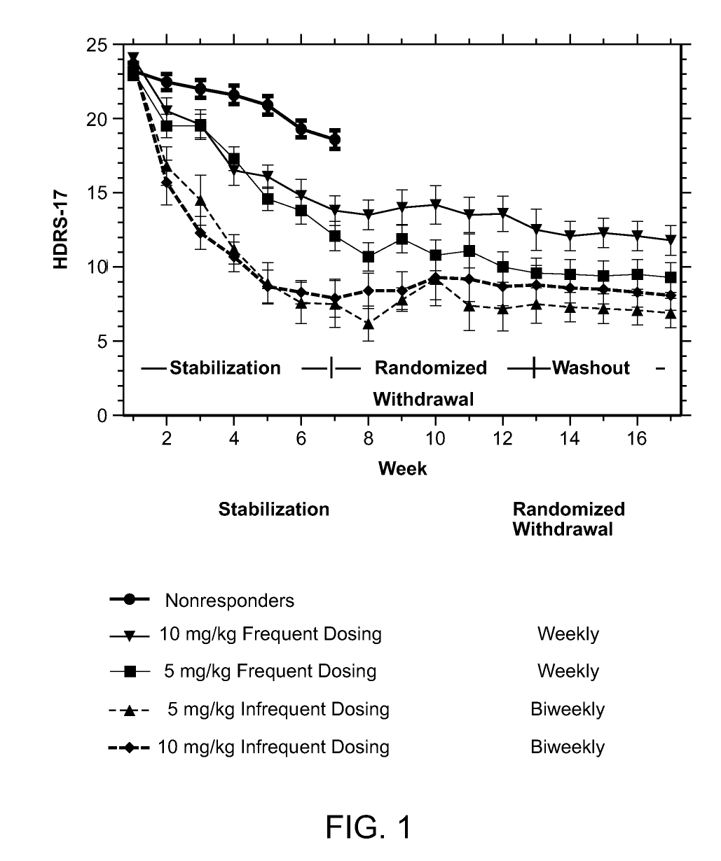

FIG. 1 is a graph showing improvements (based on HDRS scale) achieved during an induction period of time (identified as “stabilization” on the graph) are generally maintained throughout the rest period of time (identified as “randomized withdrawal” on the graph), although HDRS scores did vary with dosage frequency.

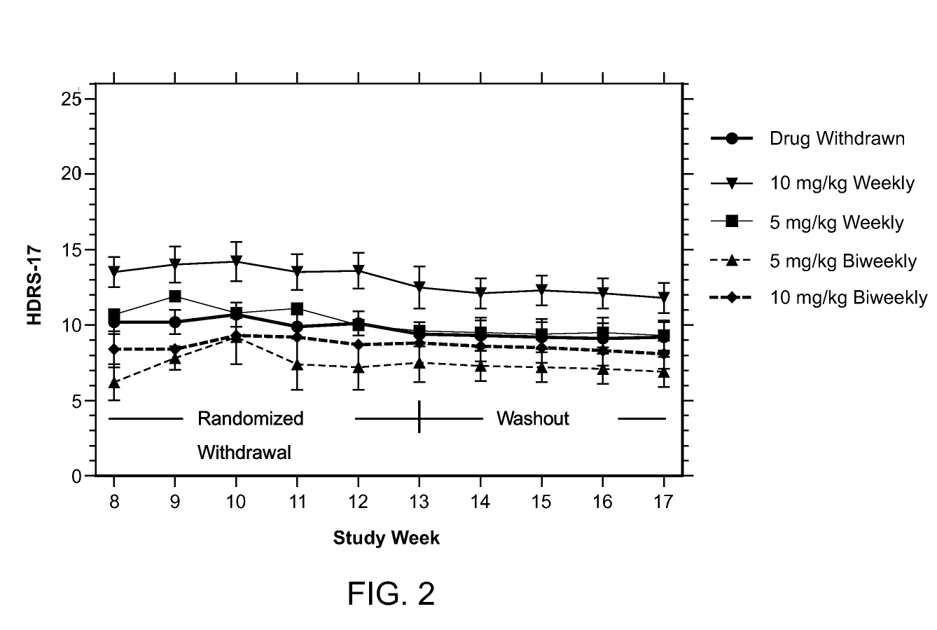

FIG. 2 is a graph showing a sub-set of data from the graph shown in FIG. 1 and shows that data for subject with drug withdrawn appear to show long-lasting effect of GLYX-13.

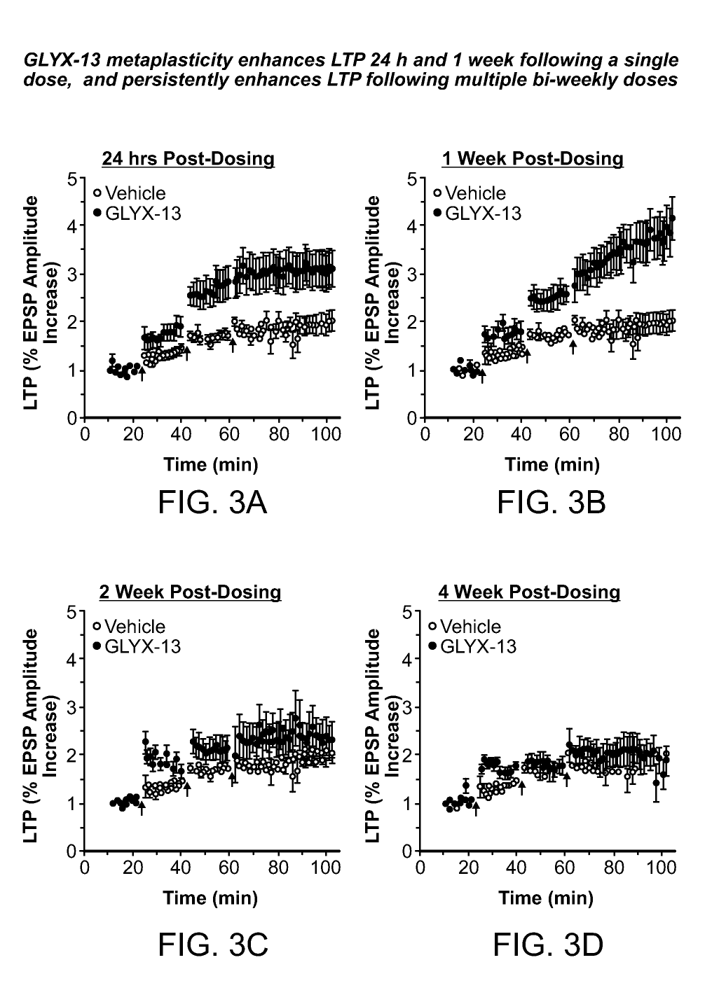

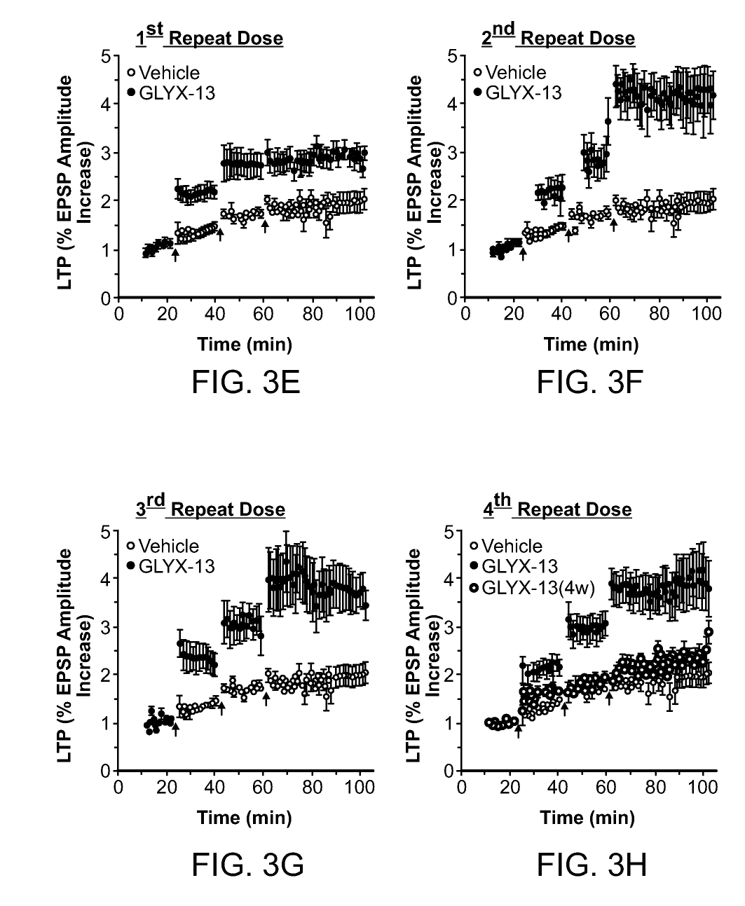

FIG. 3 is a series of graphs showing that GLYX-13 metaplasticity enhances long-term potentiation (“LTP”) 24 hours and one week following a single dose, and persistently enhances LTP following multiple bi-weekly doses.

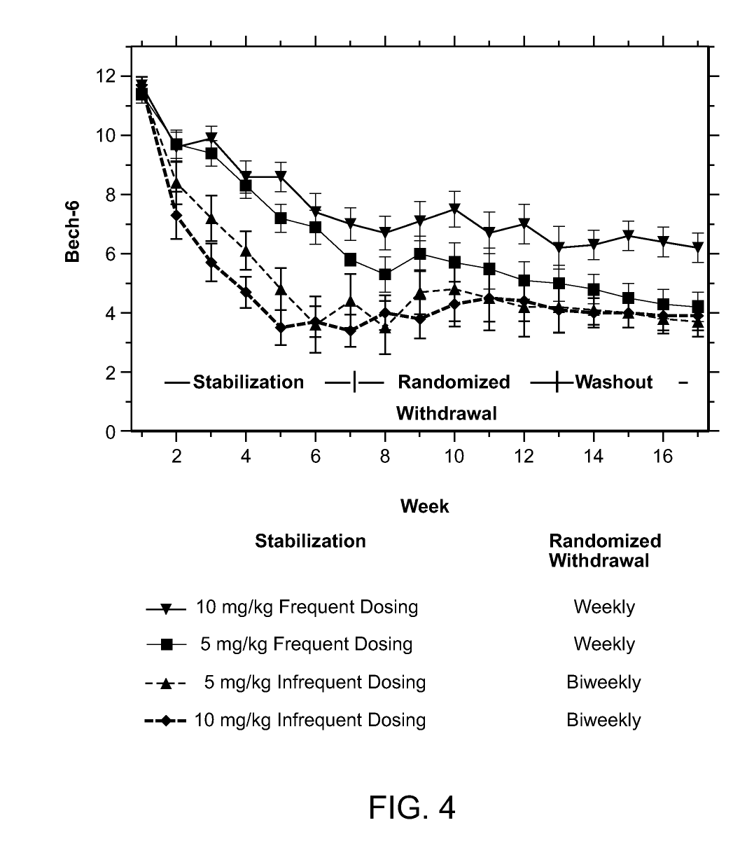

FIG. 4 is a graph showing improvements (based on Bech-6 scale) achieved during an induction period of time (identified as “stabilization” on the graph) are generally maintained throughout the rest period of time (identified as “randomized withdrawal” on the graph), although Bech-6 scores did vary with dosage frequency.

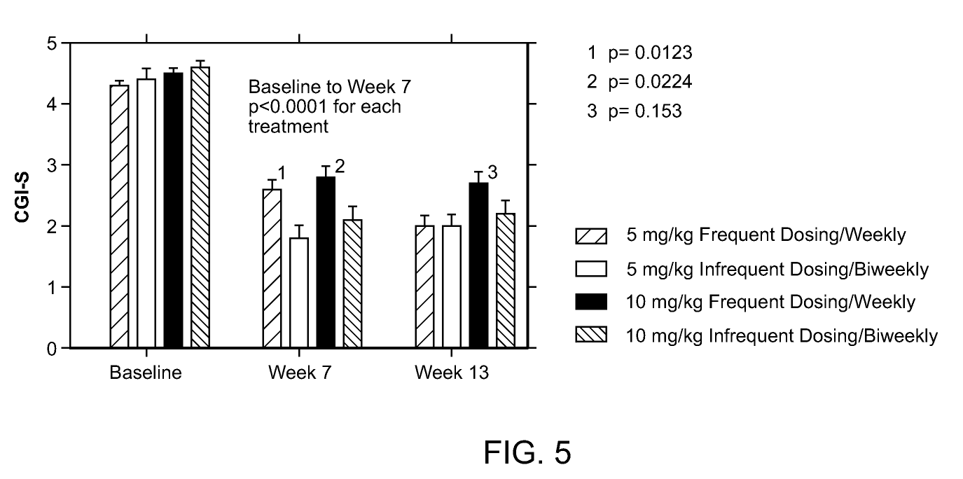

FIG. 5 is a graph showing improvements (based on CGI-S scale) achieved during an induction period of time are generally maintained throughout the rest period of time (identified as “Week 7” and “Week 13” on the graph), although CGI-S scores did vary with dosage frequency.

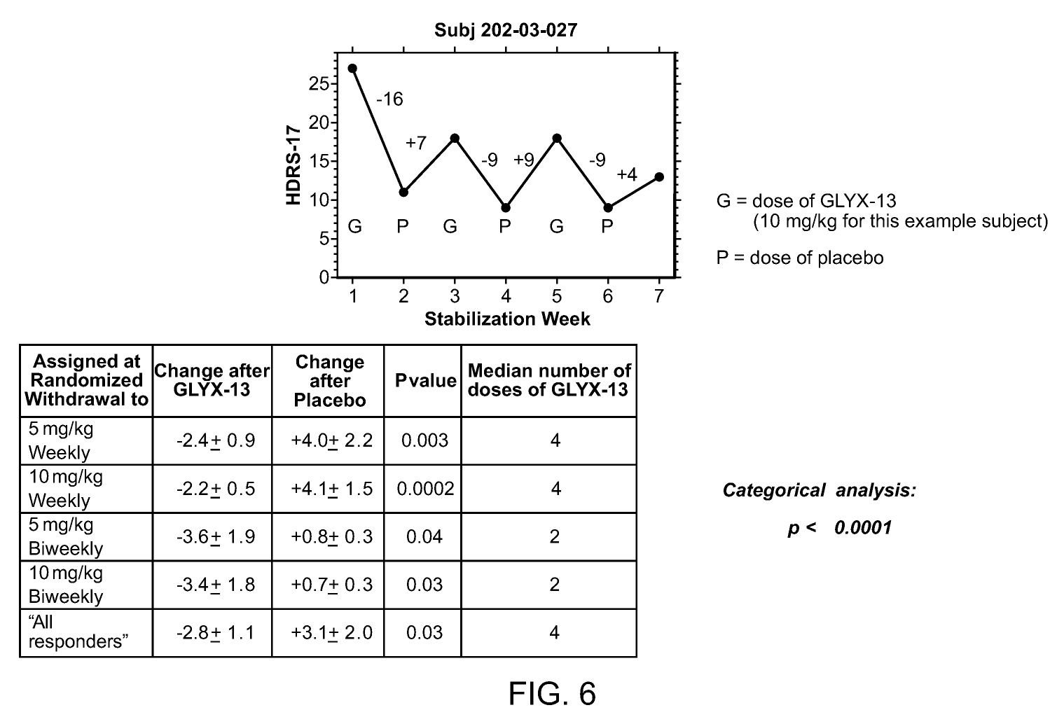

FIG. 6 is a graph showing that efficacy of GLYX-13 was reestablished following forced relapse. Repeated crossover demonstrated repeated response to GLYX-13 and relapse to placebo.

This study indicated that repeated dosing of GLYX-13 results in long-lasting efficacy without significant side effect, and with no tolerance apparent with repeated dosing. Efficacy was reestablished following forced relapse, with repeated crossover study demonstrating repeated response to GLYX-13 and relapse to placebo, with about 53% of subjects reaching clinical response. About two-thirds of responders relapsed quickly during early treatment, and the response became long-lasting with repeated doses. About one-third of responders relapsed slowly, even during early treatment. Both the blinded third party raters (HDRS-17) and clinicians blinded to treatment group made identical relative assignments to efficacy.

### Example 2 Repeat Dose Study

A repeat dose study, as reflected in Example 1 was conducted on human subjects using adjunctive dosing (patients were currently taking another antidepressant medication).

Methods. GLYX-13 was administered over 12 weeks to subjects who had responded inadequately to another antidepressant agent. Subjects continued taking the other antidepressant during the entire course of the study. The study was divided into 3 parts. During the first 6 weeks, subjects were randomized to receive GLYX-13 at 5 mg/kg IV or 10 mg/kg IV. Subjects returned to the clinic weekly. If subjects had reached clinical response (HDRS-17 reduced ≧50% from baseline) to GLYX-13 administered the previous week, placebo was administered weekly until relapse (HDRS-17 reduced <50% from baseline). At the 6^{th} week of evaluation, subjects who had achieved response to GLYX-13 at some visit were assigned to weekly or biweekly dosing based on time to relapse during placebo administration and randomized to continue receiving GLYX-13 or placebo (randomized withdrawal) for a subsequent 6 weeks of dosing. At the end of the randomized withdrawal period, subjects received placebo injections for 4 weeks. Subjects were blind to the treatments. Third party evaluators blind to treatment and protocol were utilized. Treatment dose and interval were calculated by an interactive web-based response system based on a mathematical algorithm; site personnel were blinded to treatment.

Of the subjects, 67% were female, 33% male, median age was 50 years, with median diagnosis of MDD 18 years on average. Baseline HDRS-17 score was 23.2-24.1 across groups. During the 6 week adaptive dose interval period 53% of 368 subjects reached response. Approximately 67% of responders relapsed within 2 weeks following cessation of GLYX-13 and were assigned to weekly dosing during the randomized withdrawal period whereas 33% of subjects relapsed more slowly and were assigned to biweekly dosing. At the end of the randomized withdrawal period, 65% of subjects who received biweekly doses of GLYX-13 achieved response and 45% achieved remission (defined as HDRS score ≦7). Percent of subjects who achieved remission in the 5 mg/kg weekly group was not statistically different from the biweekly dose groups whereas only 42% of subjects who received 10 mg/kg weekly reached remission. Site investigators blinded to treatment groups and dose level also evaluated subjects using the CGI-S. CGI-I at baseline was 4.3-4.6 across groups. At the end of the randomized withdrawal period, CGI-I scores were reduced by 2.5±0.12 in the biweekly groups 2.5+0.14 in the 5 mg/kg IV weekly dose group, but was reduced only 1.6±0.15 in the 10 mg/kg IV group. HDRS-17 scores did not return toward baseline in the subjects randomized to placebo during the second 6 week period, and during the 4 week washout phase following the randomized withdrawal phase, HDRS-17 scores remained at their low level in subjects who had been receiving GLYX-13 as well as the subjects who had been receiving placebo. Thus, in subjects who received placebo for 10 weeks after attaining response to GLYX-13 maintained low HDRS-17 scores.

Adjunctive GLYX-13 caused reduction in HDRS-17 scores in subjects with MDD that had responded inadequately to another antidepressant agent. Following the first few doses, response relapsed over a week or more but as treatment continued, decrease in HDRS-17 in response to each dose of GLYX-13 progressively decreased such that following 6 weeks of dosing scores were reduced from 23.5±0.34 at baseline to 10.3±0.65 in responders. Maximum reduction of HDRS-17 scores in all dosing groups was apparent by week 10 (week 3 of randomized withdrawal). Following 6 weeks of dosing, withdrawal of GLYX-13 was not associated with return of HDRS-17 score toward baseline for up to 10 weeks.

All data are expressed as mean±S.E.M. Exploration data are analysed by a two-way analysis of variance (ANOVA). This detects the main effect of drug treatment, the main effect of task, and the interaction between drug treatment and object exploration. When a significant effect is found, further analysis by a post hoc Student's t-test is performed to compare the times spent exploring the novel and familiar object. The primary endpoint is the discrimination index (DI). The DI (novel-familiar/novel+familiar) data are analysed using one-way ANOVA followed by the Bonferroni test when a significant effect was detected by the ANOVA.

Those skilled in the art will recognize, or be able to ascertain using no more than routine experimentation, many equivalents to the specific embodiments of the invention described herein. Such equivalents are intended to be encompassed by the following claims.

The entire contents of all patents, published patent applications, websites, and other references cited herein are hereby expressly incorporated herein in their entireties by reference.

1. A method of stabilizing a patient being treating for depression comprising intravenously administering to the patient an effective amount of a composition comprising GLYX-13, wherein the composition is administered to the patient once every week or once every two weeks for an induction period of time.

2. The method of claim 1, wherein the patient being treated for depression is being administered another anti-depressant without achieving a full response to treatment on the other anti-depressant alone.

3. The method of claim 1, wherein the induction period of time is about five weeks to about eight weeks, or about three weeks to about twelve weeks or more.

4. The method of claim 1, wherein the induction period of time is about six weeks.

5. The method of claim 1, wherein the after the induction period of time, the composition comprising GLYX-13 is not administered to the patient for a rest period of time of time.

6. The method of claim 2, wherein the patient is administered the other anti-depressant during the rest period of time.

7. The method of claim 1, wherein the induction period of time is three weeks, four weeks, five weeks, two months, or more.

8. The method of claim 5, wherein during the rest period of time of time, the patient substantially maintains the improvement of depression symptoms as compared to the improvement after the induction period of time as indicated by the HDRS-17 scale.

9. The method of claim 8, wherein during the rest period of time of time, the patient substantially maintains the improvement of depression symptoms by administration of the other antidepressant as compared to the improvement after the induction period of time as indicated by the HDRS-17 scale.

10. The method of claim 5, wherein during the rest period of time of time, the patient has a HDRS-17 score of less than or about 7.

11. The method of claim 5, wherein during the rest period of time of time, the patient substantially maintains the improvement of depression symptoms as compared to the improvement after the induction period of time as indicated by the MADRS scale.

12. The method of claim 11, wherein during the rest period of time of time, the patient has a MADRS score of less than or about 10.

13. The method of claim 1, wherein immediately after the induction period and during the rest period of time of time, the patient maintains a less than or equal to about 50% reduction in depression symptoms.

14. The method of claim 1, wherein the patient is also being administered another anti-depressant agent before, during or after the induction period.

15. A method of treating depression in a patient need thereof, comprising sequentially administering to the patient between about 2.5 mg/kg and about 10 mg/kg of GLYX-13 for a first period of time, and wherein the sequentially administrating is followed by not administering GLYX-13 for a rest period of time of time.

16. The method of claim 15, wherein the sequentially administrating and not administering are repeated at least once.

17. The method of claim 15, wherein the GLYX-13 is administered weekly or every other week for a first period of time of about three weeks to about twelve weeks, and wherein the rest period of time of time is about one to about six weeks or more.

18. The method of claim 15, comprising sequentially administering about 5 mg/kg or about 10 mg/kg weekly or every other week for six to twelve weeks.

19. The method of claim 15, wherein the patient is also being administered another antidepressant agent before and/or during or after the first period of time.

20. The method of claim 15, wherein the patient is being administered another antidepressant agent during the rest period of time of time.

21. A regimen for treating depression in a human patient, said regimen comprising delivering to the patient GLYX-13 in a cycle of treatment, said cycle comprising intravenously administering a dosage of about 225 mg to about 900 mg of GLYX-13 per week or every other week for at least four weeks in the cycle followed by at least one week, two weeks, three weeks, four weeks, two months or more where no GLYX-13 is administered.

22. The regimen according to claim 16, wherein the cycle comprises at least three weekly dosage administrations.

23. The regimen according to claim 19, wherein the cycle is repeated at least once.

24. The regimen according to claim 21, wherein the cycle is repeated for two to twelve cycles.

25. The regimen according to claim 21, wherein the cycles are continuous.

26. The regimen according to claim 21, wherein the patient is also being administered another antidepressant agent before and/or during the cycle.

27. The method of claim 1 wherein the depression is treatment-resistant depression.

28. The method of claim 27, wherein the treatment-resistant patient is identified as one who has been treated with at least one type of antidepressant treatments prior to administration of GLYX-13.

29. The method of claim 27, wherein the treatment-resistant patient is identified as one who has been treated with at least two types of antidepressant treatments prior to administration of GLYX-13.

30. The method of claim 1, wherein said depression is selected from the group consisting of major depressive disorder, dysthymic disorder, psychotic depression, postpartum depression, seasonal affective disorder, bipolar disorder, bipolar depression, mood disorder, depressions caused by chronic medical conditions such as cancer or chronic pain, chemotherapy, chronic stress, post traumatic stress disorders, and manic depressive disorder.

31. The method of claim 15 wherein the depression is treatment-resistant depression.

32. The method of claim 31, wherein the treatment-resistant patient is identified as one who has been treated with at least one type of antidepressant treatments prior to administration of GLYX-13.

33. The method of claim 31, wherein the treatment-resistant patient is identified as one who has been treated with at least two types of antidepressant treatments prior to administration of GLYX-13.

34. The method of claim 15, wherein said depression is selected from the group consisting of major depressive disorder, dysthymic disorder, psychotic depression, postpartum depression, seasonal affective disorder, bipolar disorder, bipolar depression, mood disorder, depressions caused by chronic medical conditions such as cancer or chronic pain, chemotherapy, chronic stress, post traumatic stress disorders, and manic depressive disorder.

35. The method of claim 21 wherein the depression is treatment-resistant depression.

36. The method of claim 35, wherein the treatment-resistant patient is identified as one who has been treated with at least one type of antidepressant treatments prior to administration of GLYX-13.

37. The method of claim 35, wherein the treatment-resistant patient is identified as one who has been treated with at least two types of antidepressant treatments prior to administration of GLYX-13.

38. The method of claim 21, wherein said depression is selected from the group consisting of major depressive disorder, dysthymic disorder, psychotic depression, postpartum depression, seasonal affective disorder, bipolar disorder, bipolar depression, mood disorder, depressions caused by chronic medical conditions such as cancer or chronic pain, chemotherapy, chronic stress, post traumatic stress disorders, and manic depressive disorder.
